(19) United States
(12) Patent Application Publication
(10) Pub. No.: US 2024/0307341 A1
(43) Pub. Date: Sep. 19, 2024

(54) Treatment of COVID-19 and Associated Pathologies

(71) Applicant: Chongcha (Fujian) Culture & Technology Co. Ltd., Fuzhou City (CN)

(72) Inventors: Feng Lin, San Diego, CA (US); Thomas Ichim, San Diego, CA (US); Jingyun Wu, Beijing (CN); Ju Zhang, Beijing (CN)

(73) Assignee: Chongcha (Fujian) Culture & Technology Co. Ltd., Fuzhou City (CN)

(21) Appl. No.: 18/603,532

(22) Filed: Mar. 13, 2024

Related U.S. Application Data
(60) Provisional application No. 63/451,920, filed on Mar. 13, 2023.

(51) Int. Cl. A61K 31/26; A61K 31/12; A61K 31/353; A61P 29/00

(52) U.S. Cl. CPC A61K 31/26; A61K 31/12; A61K 31/353; A61P 29/00

(57) ABSTRACT

Unique compositions of matter and formulations useful for treatment of COVID-19. Said formulation function by induction of immune modulation, cellular protection, suppression of inflammation and suppression of viral infection. In one embodiment a formulation consists of therapeutic doses of various nutraceuticals include epigallocatechin gallate (EGCG), sulforaphane, and curcumin. In some embodiments, the preparation are utilized as a adjuvant therapeutic, while in other embodiments said formulation is utilized as a monotherapy. Various delivery mechanisms can be used, including oral, transdermal, sublingual, intrarectal and intravenous administration.

### CROSS-REFERENCE TO RELATED APPLICATIONS

The present application claims benefit of U.S. Provisional Patent Application Ser. No. 63/451,920, filed on March 13th, 2023, entitled “TREATMENT OF COVID-19 AND ASSOCIATED PATHOLOGIES”, the contents of which are incorporated herein by reference in its entirety.

### FIELD OF THE INVENTION

The teachings herein relate to the use of nutraceuticals for treating COVID-19.

### BACKGROUND OF THE INVENTION

COVID-19 is an infectious disease caused by the coronavirus SARS-COV-2, which was first identified in December 2019 in Wuhan, China. It can cause a wide range of symptoms, from mild to severe, including fever, cough, fatigue, shortness of breath, loss of taste or smell, body aches, and sore throat. COVID-19 spreads mainly through respiratory droplets when an infected person talks, coughs, or sneezes, and can also be transmitted by touching a surface contaminated with the virus and then touching the face. COVID-19 has led to a global pandemic and has had a significant impact on public health, the economy, and daily life around the world. COVID-19 can affect anyone who is exposed to the virus. However, some groups of people may be more susceptible to severe illness or complications from COVID-19. This includes: Older adults (over age 60); People with underlying medical conditions, such as heart disease, lung disease, diabetes, or a weakened immune system; Pregnant women People who are obese;

People who smoke; People who live or work in close quarters, such as nursing home residents, prison inmates, and healthcare workers; and People from certain racial or ethnic groups, who may be at higher risk due to underlying health disparities and social determinants of health.

COVID-19 is caused by the SARS-COV-2 virus and can lead to a wide range of symptoms, including inflammation. The exact mechanisms by which the virus causes inflammation are not fully understood, but there are a few ways in which the virus can trigger an inflammatory response in the body; a) Immune response: When the SARS-COV-2 virus enters the body, the immune system is activated to fight off the infection. This response can trigger the release of cytokines, which are signaling molecules that help regulate the immune response. In some people, the immune response can become overactive and lead to a condition called a cytokine storm, which can cause widespread inflammation and tissue damage; b) Direct damage to cells: The SARS-COV-2 virus can directly infect and damage cells in the body, particularly in the lungs. This damage can trigger an inflammatory response as the body tries to repair the damaged tissue; and c) Blood clotting: COVID-19 can also cause blood clots to form in the body, which can lead to inflammation. The formation of blood clots can trigger an immune response and cause damage to the blood vessels, which can lead to inflammation and tissue damage.

Overall, COVID-19 can cause inflammation in several ways, including through the immune response, direct damage to cells, and blood clotting. The inflammation caused by COVID-19 can lead to a range of symptoms, including fever, cough, and difficulty breathing, and can also contribute to more severe complications, such as acute respiratory distress syndrome (ARDS) and organ failure. The treatment for COVID-19 (the disease caused by the SARS-COV-2 virus) can vary depending on the severity of the illness and the specific symptoms that a patient presents. Treatment may also differ depending on the country or region where a patient receives care. Mild cases of COVID-19 typically do not require any specific treatment and can be managed with rest, hydration, and over-the-counter medications such as acetaminophen or ibuprofen to relieve symptoms like fever, cough, and body aches. For more severe cases, treatment may include oxygen therapy to help with breathing difficulties, antiviral medications such as remdesivir or monoclonal antibodies, and anti-inflammatory drugs such as corticosteroids to reduce inflammation in the lungs. In some cases, patients may require mechanical ventilation or extracorporeal membrane oxygenation (ECMO) to support their breathing.

At present there are no established means of treating COVID-19 that possess repeated success in widespread clinical trials. Furthermore, there is a need to prevent the long term effects of COVID-19 pathology including pulmonary and neurological damage as well as suppression of various other organ functions. The current invention provides means of suppressing

### SUMMARY OF THE INVENTION

At least one specification heading is required. Please delete this heading section if it is not applicable to your application. For more information regarding the headings of the specification, please see MPEP 608.01(a).

Preferred methods include embodiments of preventing, treating, or reducing COVID-19 infection and/or pathology associated with said infection, said method comprising: a) identifying a patient with COVID-19 or at risk of COVID-19 infection; administering a composition containing curcumin, sulforaphane and epigallocatechin gallate (EGCG)I and c) optionally adjusting dose based on clinical and/or laboratory response.

Preferred embodiments include methods wherein said COVID-19 pathology is neuroinflammation.

Preferred embodiments include methods wherein said neuroinflammation is activation of microglial cells.

Preferred embodiments include methods wherein said activated microglial produce an increase amount of TNF-alpha as compared to an unactivated microglial cell.

Preferred embodiments include methods wherein said activated microglial produce an increase amount of IL-1 beta as compared to an unactivated microglial cell.

Preferred embodiments include methods wherein said activated microglial produce an increase amount of IL-6 as compared to an unactivated microglial cell.

Preferred embodiments include methods wherein said activated microglial produce an increase amount of IL-18 as compared to an unactivated microglial cell.

Preferred embodiments include methods wherein said activated microglial produce an increase amount of IL-27 as compared to an unactivated microglial cell.

Preferred embodiments include methods wherein said activated microglial produce an increase amount of complement C5a as compared to an unactivated microglial cell.

Preferred embodiments include methods wherein said activated microglial produce an increase amount of complement C3a as compared to an unactivated microglial cell.

Preferred embodiments include methods wherein said activated microglial produce an increase amount of indolamine 2,3 dioxygenase as compared to an unactivated microglial cell.

Preferred embodiments include methods wherein said activated microglial produce an increase amount of TRANCE as compared to an unactivated microglial cell.

Preferred embodiments include methods wherein said activated microglial produce an increase amount of TRAIL as compared to an unactivated microglial cell.

Preferred embodiments include methods wherein said neuroinflammation is associated with enhance levels of NMDA receptor activation.

Preferred embodiments include methods wherein said neuroinflammation is associated with enhance levels glutaminergic signaling.

Preferred embodiments include methods wherein said neuroinflammation is associated with reduced levels of T regulatory cells in the brain environment.

Preferred embodiments include methods wherein said neuroinflammation is associated with Th17 cell activation in the central nervous system.

Preferred embodiments include methods wherein said Th17 cell activation induces microglial expression of HLA-DR.

Preferred embodiments include methods wherein said Th17 cell activation induces microglial expression of CD80.

Preferred embodiments include methods wherein said Th17 cell activation induces microglial expression of CD40.

Preferred embodiments include methods wherein said Th17 cell activation induces microglial expression of CD86.

Preferred embodiments include methods wherein said Th17 cell activation reduces microglial expression of HLA-G.

Preferred embodiments include methods wherein said Th17 cell activation induces endothelial expression of VCAM-1.

Preferred embodiments include methods wherein said Th17 cell activation induces endothelial expression of nitric oxide.

Preferred embodiments include methods wherein said Th17 cell activation induces endothelial expression NF-kappa B.

Preferred embodiments include methods wherein said Th17 cell activation induces endothelial expression of STAT3.

Preferred embodiments include methods wherein said Th17 cell activation induces endothelial expression of i-kappa B.

Preferred embodiments include methods wherein said Th17 cell activation increases ability of endothelium to allow for adhesion of inflammatory cells.

Preferred embodiments include methods wherein said inflammatory cells are CD4 T cells.

Preferred embodiments include methods wherein said CD4 T cells are Th1 cells.

Preferred embodiments include methods wherein said CD4 T cells are Th17 cells.

Preferred embodiments include methods wherein said inflammatory cells are neutrophils.

Preferred embodiments include methods wherein said neutrophils are capable of performing antigen presentation.

Preferred embodiments include methods wherein said neutrophils are capable of producing defesin B.

Preferred embodiments include methods wherein said neutrophils are N1 neutrophils.

Preferred embodiments include methods wherein said inflammatory cells are monocytes.

Preferred embodiments include methods wherein said monocytes are M1 monocytes.

Preferred embodiments include methods wherein said inflammatory cells are NK cells.

Preferred embodiments include methods wherein said inflammatory cells are NKT cells.

Preferred embodiments include methods wherein said inflammatory cells are gamma delta T cells.

Preferred embodiments include methods wherein said inflammatory cells are mast cells.

Preferred embodiments include methods wherein said inflammatory cells are telocytes.

Preferred embodiments include methods wherein said inflammatory cells are basophils.

Preferred embodiments include methods wherein said inflammatory cells are eosinophils.

Preferred embodiments include methods wherein said neuroinflammation is characterized by decreased neurogenesis.

Preferred embodiments include methods wherein said decreased neurogenesis is observed in the dentate gyrus.

Preferred embodiments include methods wherein said decreased neurogenesis is observed in the subventricular zone.

Preferred embodiments include methods wherein said decreased neurogenesis is associated with reduction of proliferation of CD133 expressing cells in the central nervous system.

Preferred embodiments include methods wherein said decreased neurogenesis is associated with reduction of proliferation of c-met expressing cells in the central nervous system.

Preferred embodiments include methods wherein said decreased neurogenesis is associated with reduction of proliferation of interleukin-3 receptor expressing cells in the central nervous system.

Preferred embodiments include methods wherein said decreased neurogenesis is associated with reduction of proliferation of BDNF receptor expressing cells in the central nervous system.

Preferred embodiments include methods wherein said decreased neurogenesis is associated with reduction of proliferation of NGF receptor expressing cells in the central nervous system.

Preferred embodiments include methods wherein said decreased neurogenesis is associated with reduction of proliferation of interleukin-6 receptor expressing cells in the central nervous system.

Preferred embodiments include methods wherein said COVID-19 associated pathology is decrease in regenerative activity of type II pulmonary epithelial cells.

Preferred embodiments include methods wherein said decrease said regenerative activity of said type 1 pulmonary epithelial cells is reduced proliferative activity in response to inflammatory stimuli.

Preferred embodiments include methods wherein said inflammatory stimuli is mediators released after cellular necrosis.

Preferred embodiments include methods wherein said inflammatory stimuli is mediators released after cellular pyroptosis.

Preferred embodiments include methods wherein said inflammatory stimuli is mediators released after cellular ferroptosis.

Preferred embodiments include methods wherein said inflammatory stimuli is mediators released after cellular apoptosis.

Preferred embodiments include methods wherein said inflammatory stimuli is HMGB1.

Preferred embodiments include methods wherein said inflammatory stimuli is TNF-

alpha.

Preferred embodiments include methods wherein said inflammatory stimuli is IL-1 beta.

Preferred embodiments include methods wherein said inflammatory stimuli is tissue factor.

Preferred embodiments include methods wherein said inflammatory stimuli is a complement component.

Preferred embodiments include methods wherein said inflammatory stimuli is small molecular weight hyaluronic acid fragments.

Preferred embodiments include methods wherein said inflammatory stimuli is a toll like receptor agonist.

Preferred embodiments include methods wherein said inflammatory stimuli is double stranded RNA.

Preferred embodiments include methods wherein said inflammatory stimuli is free DNA. Preferred embodiments include methods wherein said inflammatory stimuli is circular DNA.

Preferred embodiments include methods wherein said inflammatory stimuli is double stranded RNA.

Preferred embodiments include methods wherein said inflammatory stimuli is lipopolysaccharide.

Preferred embodiments include methods wherein said inflammatory stimuli is flagellin.

Preferred embodiments include methods wherein said inflammatory stimuli is uric acid crystals.

Preferred embodiments include methods wherein said inflammatory stimuli is interferon alpha.

Preferred embodiments include methods wherein said inflammatory stimuli is interferon beta.

Preferred embodiments include methods wherein said inflammatory stimuli is interferon gamma.

Preferred embodiments include methods wherein said inflammatory stimuli is interferon lambda.

Preferred embodiments include methods wherein said inflammatory stimuli is interferon tau.

Preferred embodiments include methods wherein said decrease in regenerative activity of type 2 pulmonary epithelial cells is characterized by increased fibrosis.

Preferred embodiments include methods wherein said decrease in regenerative activity of type 2 pulmonary epithelial cells is characterized by increased collagen deposition.

Preferred embodiments include methods wherein said decrease in regenerative activity of type 1 pulmonary epithelial cells is characterized by increased tissue inhibitor of matrix metalloproteases.

Preferred embodiments include methods wherein said decrease in regenerative activity of type 1 pulmonary epithelial cells is characterized by decreased plasminogen activity.

Preferred embodiments include methods wherein said decrease in regenerative activity of type 1 pulmonary epithelial cells is characterized by decreased MMP1 activity.

Preferred embodiments include methods wherein said decrease in regenerative activity of type 1 pulmonary epithelial cells is characterized by decreased MMP3 activity.

Preferred embodiments include methods wherein said decrease in regenerative activity of type 1 pulmonary epithelial cells is characterized by decreased MMP5 activity.

Preferred embodiments include methods wherein said decrease in regenerative activity of type 1 pulmonary epithelial cells is characterized by decreased MMP9 activity.

Preferred embodiments include methods wherein said decrease in regenerative activity of type 1 pulmonary epithelial cells is characterized by decreased MMP12 activity.

Preferred embodiments include methods wherein said decrease in regenerative activity of type 1 pulmonary epithelial cells is characterized by decreased TGF-alpha activity.

Preferred embodiments include methods wherein said decrease in regenerative activity of type 1 pulmonary epithelial cells is characterized by decreased TGF-beta activity.

Preferred embodiments include methods wherein said COVID-19 infection is associated with increase permeability of the blood brain barrier.

Preferred embodiments include methods wherein said increased permeability of said blood brain barrier allows for enhanced infiltration of inflammatory cells into the central nervous system.

Preferred embodiments include methods wherein said increased permeability of said blood brain barrier allows for decreased trafficking of T regulatory cells into the central nervous system.

Preferred embodiments include methods wherein said T regulatory cells express membrane bound TGF-beta.

Preferred embodiments include methods wherein said T regulatory cells express CTLA-4.

Preferred embodiments include methods wherein said T regulatory cells express membrane bound fas ligand.

Preferred embodiments include methods wherein said T regulatory cells express GITR. Preferred embodiments include methods wherein said T regulatory cells express FoxP3.

Preferred embodiments include methods wherein said T regulatory cells secrete IL-10.

Preferred embodiments include methods wherein said T regulatory cells are capable of inducing angiogenesis when activated with anti-CD3.

Preferred embodiments include methods wherein said T regulatory cells are capable of inducing angiogenesis when activated with anti-CD3 and anti-CD28.

Preferred embodiments include methods wherein said T regulatory cells inhibit proliferation of activated T cells.

Preferred embodiments include methods wherein said T regulatory cells inhibit differentiation of dendritic cells in response to a maturation agent.

Preferred embodiments include methods wherein said differentiation of dendritic cells is upregulation of CD40 expression.

Preferred embodiments include methods wherein said differentiation of dendritic cells is upregulation of CD80 expression.

Preferred embodiments include methods wherein said differentiation of dendritic cells is upregulation of CD86 expression.

Preferred embodiments include methods wherein said differentiation of dendritic cells is upregulation of CD5 expression.

Preferred embodiments include methods wherein said differentiation of dendritic cells is upregulation of interleukin-12 production.

Preferred embodiments include methods wherein said COVID-19 infection is associated with pulmonary fluid retention.

Preferred embodiments include methods wherein said increased pulmonary fluid retention is associated with augmented pulmonary expression of interleukin-1 beta.

Preferred embodiments include methods wherein said increased pulmonary fluid retention is associated with augmented pulmonary expression of interleukin-5.

Preferred embodiments include methods wherein said increased pulmonary fluid retention is associated with augmented pulmonary expression of interleukin-6.

Preferred embodiments include methods wherein said increased pulmonary fluid retention is associated with augmented pulmonary expression of interleukin-8.

Preferred embodiments include methods wherein said increased pulmonary fluid retention is associated with augmented pulmonary expression of interleukin-9.

Preferred embodiments include methods wherein said increased pulmonary fluid retention is associated with augmented pulmonary expression of interleukin-11.

Preferred embodiments include methods wherein said increased pulmonary fluid retention is associated with augmented pulmonary expression of interleukin-12.

Preferred embodiments include methods wherein said increased pulmonary fluid retention is associated with augmented pulmonary expression of interleukin-15.

Preferred embodiments include methods wherein said increased pulmonary fluid retention is associated with augmented pulmonary expression of interleukin-16.

Preferred embodiments include methods wherein said increased pulmonary fluid retention is associated with augmented pulmonary expression of interleukin-17.

Preferred embodiments include methods wherein said increased pulmonary fluid retention is associated with augmented pulmonary expression of interleukin-18.

Preferred embodiments include methods wherein said increased pulmonary fluid retention is associated with augmented pulmonary expression of interleukin-22.

Preferred embodiments include methods wherein said increased pulmonary fluid retention is associated with augmented pulmonary expression of interleukin-23.

Preferred embodiments include methods wherein said increased pulmonary fluid retention is associated with augmented pulmonary expression of interleukin-27.

Preferred embodiments include methods wherein said increased pulmonary fluid retention is associated with augmented pulmonary expression of interleukin-33.

Preferred embodiments include methods wherein said increased pulmonary fluid retention is associated with augmented pulmonary expression of HMGB1.

### BRIEF DESCRIPTION OF DRAWINGS

At least one specification heading is required. Please delete this heading section if it is not applicable to your application. For more information regarding the headings of the specification, please see MPEP 608.01(a).

FIG. 1 is a bar graph showing the effects of 10, 20 or 40 ug of sulforaphane in combination with Curcumin (10 mg/kg), EGCG (40 mg/kg) on TNF-alpha in a chronic lung damage murine model.

FIG. 2 is a bar graph showing the effects of 10, 20 or 40 ug of sulforaphane in combination with Curcumin (10 mg/kg), EGCG (40 mg/kg) on IL-6 in a chronic lung damage murine model.

FIG. 3 is a bar graph showing the effects of 10, 20 or 40 ug of sulforaphane in combination with Curcumin (10 mg/kg), EGCG (40 mg/kg) on IL-8 in a chronic lung damage murine model.

FIG. 4 is a bar graph showing the effects of 10, 20 or 40 ug of sulforaphane in combination with Curcumin (10 mg/kg), EGCG (40 mg/kg) on HMGB-1 in a chronic lung damage murine model.

### DETAILED DESCRIPTION OF THE INVENTION

The invention provides the unexpected finding that administration of nutraceutical mixtures containing curcumin, EGCG, pterostilbene and sulforaphane possess ability to suppress inflammation of the lung and pathology associated thereof. In one embodiment, the invention teaches that administration of said ingredients, along, or in combination, is capable of suppressing SARS-COV-2 spike protein induced lung inflammation. In some embodiments the patent teaches administration of curcumin, EGCG, pterostilbene and sulforaphane is capable of downregulating inflammatory cytokine production associated with lung inflammation similar to that observed in COVID-19 infection and long term follow up.

In some embodiments the present invention includes a method of treating or preventing complications associated with a SARS-COV-2 infection, Rhinovirus, and mRNA vaccines comprising administration of a combination of Nutraceutical compositions of various combinations of cannabinoids, vitamins, trace elements, bioactive components, bioflavonoid polyphenols, proteolytic enzymes, amino acids, and antioxidants with at least one combination of 3 or more of Glutathione, N-acetylcysteine, Alpha Lipoic Acid, Quercetin, Zinc, Vitamin D3, Vitamin K2, Vitamin B6, Vitamin B9, Vitamin B12, Magnesium, Bromelain, Cannabidiolic Acid, Cannabigerolic Acid, Cannabidiol, curcumin, Cannabigerol, Cannabinol, Nigella Sativa/Thymoquinone, Selenium, Curcumin, Piperine, Astaxanthin, and Sulforaphane each present in therapeutically effective amounts, in an admixture with nutraceutically acceptable excipient.

According to various embodiments of the present teachings, the nutraceutical composition can comprise Cannabidiolic Acid, Cannabigerolic Acid, Cannabidiol, Cannabigerol, Vitamin D3, Vitamin K2, Magnesium, Quercetin, Zinc, Bromelain, Nigella Sativa/Thymoquinone, and Astaxanthin. According to other embodiments of the present teachings, the nutraceuticals Cannabidiolic Acid, Cannabigerolic Acid, Vitamin D3, Quercetin, Bromelain, Zinc, and Nigella Sativa/Thymoquinone can act to block COVID spike proteins and mRNA vaccine spike proteins from attaching to the ACE2 receptors on body organ cells.

The nutraceuticals Quercetin, Bromelain, Nigella Sativa/Thymoquinone, and Zinc can work synergistically and/or independently to penetrate body organ cell membranes and block the replication of virus particles and mRNA spike protein replication.

In some embodiments, EGCG, sulforaphane, pterostilbene and curcumin are administered with nutraceuticals such as N-acetylcysteine, Vitamin D3, Vitamin K2, Vitamin B6, Vitamin B12, Magnesium, Glutathione, Alpha Lipoic Acid, Selenium, Zinc, and Astaxanthin as working synergistically and/or independently to inhibit excess inflammation. Also disclosed by the present application is treatment of long-term diseases caused by the COVID virus, Rhinoviruses, and/or mRNA vaccine spike protein replication inside the cells by the synergistic and/or independent work of the nutraceuticals N-acetylcysteine, Vitamin D3, Vitamin K2, Vitamin B6, Vitamin B9, Vitamin B12, Magnesium, Glutathione, Alpha Lipoic Acid, Selenium, Zinc, and Astaxanthin. The nutraceutical Glutathione can act as both a zinc ionophore to increase the bioavailability of zinc assisting its penetration through cell membranes where the Zinc can block virus particle and mRNA spike protein replication, and as an anti-coagulant to prevent blood clots caused by the mRNA vaccines.

In addition, these unique combinations of compounds inhibit inflammation and prevent long-term side effects (long COVID, health issues, long-haulers), and disease caused by the SARS-CoV-2 and its variants, Rhinovirus, other viruses, bacteria, and mRNA vaccines caused by damage done to body organ cell tissue.

In some embodiments of the invention, certain of these antioxidants can cross the blood-brain barrier to inhibit cytokine, viral spike protein, and mRNA vaccine spike protein damage done to cells in the brain and the central nervous system. Multiple combinations of these compounds work synergistically to increase the efficacy of independent nutraceuticals targeting the inhibition of excess cytokines, COVID spike proteins, mRNA vaccine spike proteins, and inflammation. Moreover, each individual's immune system is unique, and this disclosure uses multiple nutraceuticals and formulations to cover the broadest possible range of effective inhibition excess cytokines, COVID spike proteins and mRNA spike proteins from attaching to human body organ cells, penetrating cell membranes, and replicating inside the cells. Prevention and treatment require inhibiting excess cytokines, and blocking cell attachment, penetration, and replication by SARS-COV-2 spike proteins and its variants, and/or mRNA spike proteins.

Nutraceuticals include dietary supplements. The term “dietary supplement” refers to a wide range of products including vitamins and minerals, herbs and other botanicals, amino acids, enzymes, and more.

SARS-COV-2 is a member of the class of positive-strand RNA viruses, which means that they code directly for the proteins that the RNA encodes, rather than requiring a copy to an antisense strand prior to translation into protein. The virus consists primarily of the single-strand RNA molecule packaged up inside a protein coat, consisting of the virus's structural proteins, most notably the spike protein, which facilitates both viral binding to a receptor (in the case of SARS-COV-2 this is the ACE2 receptor) and virus fusion with the host cell membrane. The SARS-COV-2 spike protein is the primary target for neutralizing antibodies. Experimental mRNA vaccines have been heralded as having the potential for great benefits, but they also harbor the possibility of potentially tragic and even catastrophic unforeseen consequences. The mRNA vaccines against SARS-COV-2 have been implemented with great fanfare, but there are many aspects of their widespread utilization that merit concern. We want to emphasize that these concerns are potentially serious and might not be evident for years or even transgenerationally.

Furthermore, as an obvious but tragically ignored suggestion, the government should also be encouraging the population to take safe and affordable steps to boost their immune systems naturally, such as getting out in the sunlight to raise vitamin D levels (Ali, 2020), and eating mainly organic whole foods rather than chemical-laden processed foods (Rico-Campà et al., 2019). Also, eating foods that are good sources of vitamin A, vitamin C and vitamin K2 should be encouraged, as deficiencies in these vitamins are linked to bad outcomes from COVID-19 (Goddek, 2020; Sarohan, 2020). (Seneff, 2021).

This comprehensive presentation of the damage being done by SARS-COV-2 spike proteins and mRNA vaccine spike proteins encompasses most severe health issues being caused by them and indicates how imperative it is to find methods of defense against the effects from exposure to SARS-COV-2 spike proteins and mRNA vaccine spike proteins. Furthermore, the mRNA vaccine instructs the immune system to continuously produce spike proteins and current research indicates that can be anywhere from 4-8 weeks and up to a year. With the advent of continuous booster shots, this is a problem that needs to be solved. The critical point at which the process of infecting cells that allow virus particle and spike protein replication begins when the SARS-COV-2 spike proteins and mRNA vaccine spike proteins attach themselves to the ACE2 receptors on body organ cells. The key to preventing severe disease and potential death from exposure to the SARS-COV-2 spike proteins and mRNA vaccine spike proteins is to block spike protein cell attachment, penetration, and replication. This disclosure proposes the use of nutraceuticals to inhibit the SARS-COV-2 spike proteins and mRNA vaccine spike proteins from attaching to the ACE2 receptors of body organ cells such as the brain, kidney, liver, heart, lungs, and other body organ cells. Blocking the SARS-COV-2 spike proteins and mRNA vaccine spike proteins from attaching to the ACE2 receptors interrupts the pathogenesis of SARS-COV-2 spike proteins and blocks entry of mRNA vaccine spike proteins into the cells where they replicate, damage the mitochondria, and cause hyperinflammation leading to long-term disease.

### Example 1: Reduction of Pulmonary Inflammation by Administration of Nutraceutical with Increasing Sulforaphane Concentration in Chronic Lung Damage Model: TNF-alpha

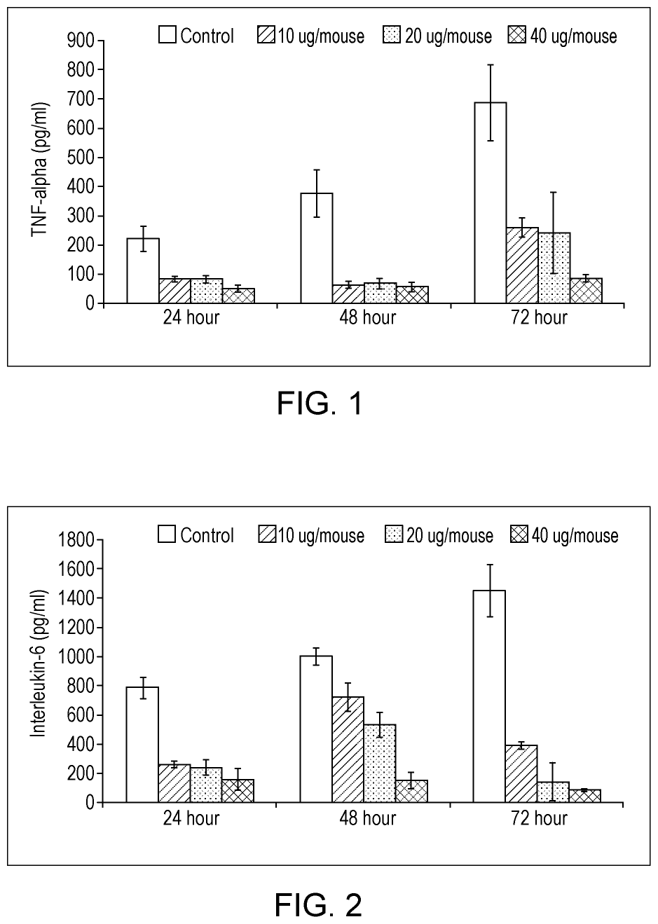

Male 7-week-old C57BL/6 mice were housed in the laboratory animal center under 22±2° C., 55% +10% humidity, and 12 h dark/light cycle conditions. All of the mice were randomized before the experiment. The model was established by intratracheal (IT) instillation of 0.2 IU porcine pancreatic elastase (Sigma-Aldrich, St. Louis, MO, USA) using a microsprayer aerosolizer (Penn-Century, Wyndmoor, PA, USA) under general anesthesia with 3% isoflurane using a rodent anesthesia machine. Prior to administration of elastase, 10, 20 or 40 ug of sulforaphane was administered along with Curcumin (10 mg/kg), EGCG (40 mg/kg) to the mice by gavage daily for 1 week. Animals were sacrificed at the indicated timepoints. Lungs were homogenized and assessed for TNF-alpha by ELISA. Results are shown in FIG. 1 and below.

Raw Data

| | | 10 ug/ | 20 ug/ | 40 ug/ |
|---|---|---|---|---|
| | Control | mouse | mouse | mouse |
| | 24 hours | | | |
| 1.0 | 234.0 | 104.0 | 76.0 | 55.0 |
| 2.0 | 232.0 | 95.0 | 78.0 | 45.0 |
| 3.0 | 225.0 | 89.0 | 99.0 | 42.0 |
| 4.0 | 231.0 | 78.0 | 85.0 | 45.0 |
| 5.0 | 204.0 | 78.0 | 67.0 | 47.0 |
| 6.0 | 157.0 | 67.0 | 86.0 | 54.0 |
| 7.0 | 256.0 | 68.0 | 88.0 | 76.0 |
| 8.0 | 215.0 | 87.0 | 78.0 | 54.0 |
| 9.0 | 235.0 | 98.0 | 98.0 | 35.0 |
| 10.0 | 226.0 | 79.0 | 87.0 | 67.0 |
| Average | 221.5 | 84.3 | 84.2 | 52.0 |
| STDEV | 26.4 | 12.4 | 9.9 | 12.2 |
| | 48 hours | | | |
| 1.0 | 224.0 | 45.0 | 76.0 | 54.0 |
| 2.0 | 323.0 | 46.0 | 77.0 | 56.0 |
| 3.0 | 443.0 | 43.0 | 75.0 | 43.0 |
| 4.0 | 346.0 | 46.0 | 65.0 | 45.0 |
| 5.0 | 326.0 | 75.0 | 45.0 | 76.0 |
| 6.0 | 358.0 | 75.0 | 75.0 | 45.0 |
| 7.0 | 357.0 | 87.0 | 88.0 | 76.0 |
| 8.0 | 489.0 | 76.0 | 75.0 | 34.0 |
| 9.0 | 435.0 | 88.0 | 67.0 | 76.0 |
| 10.0 | 474.0 | 67.0 | 54.0 | 76.0 |
| Average | 377.5 | 64.8 | 69.7 | 58.1 |
| STDEV | 81.9 | 18.1 | 12.5 | 16.5 |
| | 72 hours | | | |
| 1.0 | 554.0 | 235.0 | 213.0 | 97.0 |
| 2.0 | 765.0 | 434.0 | 223.0 | 87.0 |
| 3.0 | 567.0 | 136.0 | 215.0 | 95.0 |
| 4.0 | 887.0 | 176.0 | 254.0 | 78.0 |
| 5.0 | 768.0 | 135.0 | 275.0 | 68.0 |
| 6.0 | 875.0 | 136.0 | 284.0 | 98.0 |
| 7.0 | 673.0 | 121.0 | 286.0 | 75.0 |
| 8.0 | 648.0 | 435.0 | 216.0 | 67.0 |
| 9.0 | 558.0 | 323.0 | 196.0 | 98.0 |
| 10.0 | 576.0 | 457.0 | 263.0 | 76.0 |
| Average | 687.1 | 258.8 | 242.5 | 86.0 |
| STDEV | 129.4 | 140.3 | 33.5 | 12.5 |

### Example 2: Reduction of Pulmonary Inflammation by Administration of Nutraceutical with Increasing Sulforaphane Concentration in Chronic Lung Damage Model: Interleukin-6

Male 7-week-old C57BL/6 mice were housed in the laboratory animal center under 22±2° C., 55%±10% humidity, and 12 h dark/light cycle conditions. All of the mice were randomized before the experiment. The model was established by intratracheal (IT) instillation of 0.2 IU porcine pancreatic elastase (Sigma-Aldrich, St. Louis, MO, USA) using a microsprayer aerosolizer (Penn-Century, Wyndmoor, PA, USA) under general anesthesia with 3% isoflurane using a rodent anesthesia machine. Prior to administration of elastase, 10, 20 or 40 ug of sulforaphane was administered along with Curcumin (10 mg/kg), EGCG (40 mg/kg) to the mice by gavage daily for 1 week. Animals were sacrificed at the indicated timepoints. Lungs were homogenized and assessed for interleukin-6 by ELISA. Results are shown in FIG. 2 and below.

Raw Data

| | | 10 ug/ | 20 ug/ | 40 ug/ |
|---|---|---|---|---|
| | Control | mouse | mouse | mouse |
| | 24 hours | | | |
| 1.0 | 756.0 | 354.0 | 253.0 | 115.0 |
| 2.0 | 865.0 | 356.0 | 221.0 | 126.0 |
| 3.0 | 774.0 | 213.0 | 264.0 | 106.0 |
| 4.0 | 768.0 | 264.0 | 231.0 | 324.0 |
| 5.0 | 764.0 | 236.0 | 237.0 | 231.0 |
| 6.0 | 678.0 | 243.0 | 275.0 | 126.0 |
| 7.0 | 734.0 | 216.0 | 212.0 | 216.0 |
| 8.0 | 752.0 | 246.0 | 215.0 | 124.0 |
| 9.0 | 931.0 | 224.0 | 247.0 | 112.0 |
| 10.0 | 843.0 | 265.0 | 253.0 | 112.0 |
| Average | 786.5 | 261.7 | 240.8 | 159.2 |
| STDEV | 72.9 | 52.2 | 21.2 | 73.2 |
| | 48 hours | | | |
| 1.0 | 1002.0 | 768.0 | 554.0 | 121.0 |
| 2.0 | 969.0 | 745.0 | 565.0 | 112.0 |
| 3.0 | 996.0 | 786.0 | 764.0 | 97.0 |
| 4.0 | 964.0 | 735.0 | 456.0 | 67.0 |
| 5.0 | 964.0 | 637.0 | 547.0 | 123.0 |
| 6.0 | 979.0 | 874.0 | 542.0 | 215.0 |
| 7.0 | 942.0 | 674.0 | 452.0 | 213.0 |
| 8.0 | 1086.0 | 768.0 | 575.0 | 217.0 |
| 9.0 | 986.0 | 584.0 | 457.0 | 197.0 |
| 10.0 | 1121.0 | 653.0 | 435.0 | 175.0 |
| Average | 1000.9 | 722.4 | 534.7 | 153.7 |
| STDEV | 57.4 | 85.3 | 97.0 | 55.9 |
| | 72 hours | | | |
| 1.0 | 1436.0 | 231.0 | 197.0 | 97.0 |
| 2.0 | 1326.0 | 236.0 | 125.0 | 78.0 |
| 3.0 | 1542.0 | 221.0 | 121.0 | 84.0 |
| 4.0 | 1347.0 | 543.0 | 147.0 | 78.0 |
| 5.0 | 1744.0 | 345.0 | 121.0 | 86.0 |
| 6.0 | 1342.0 | 436.0 | 147.0 | 74.0 |
| 7.0 | 1438.0 | 432.0 | 123.0 | 83.0 |
| 8.0 | 1332.0 | 464.0 | 175.0 | 97.0 |
| 9.0 | 1754.0 | 574.0 | 157.0 | 85.0 |
| 10.0 | 1242.0 | 432.0 | 134.0 | 67.0 |
| Average | 1450.3 | 391.4 | 144.7 | 86.0 |
| STDEV | 177.2 | 128.2 | 25.6 | 9.4 |

### Example 3: Reduction of Pulmonary Inflammation by Administration of Nutraceutical with Increasing Sulforaphane Concentration in Chronic Lung Damage Model: Interleukin-8

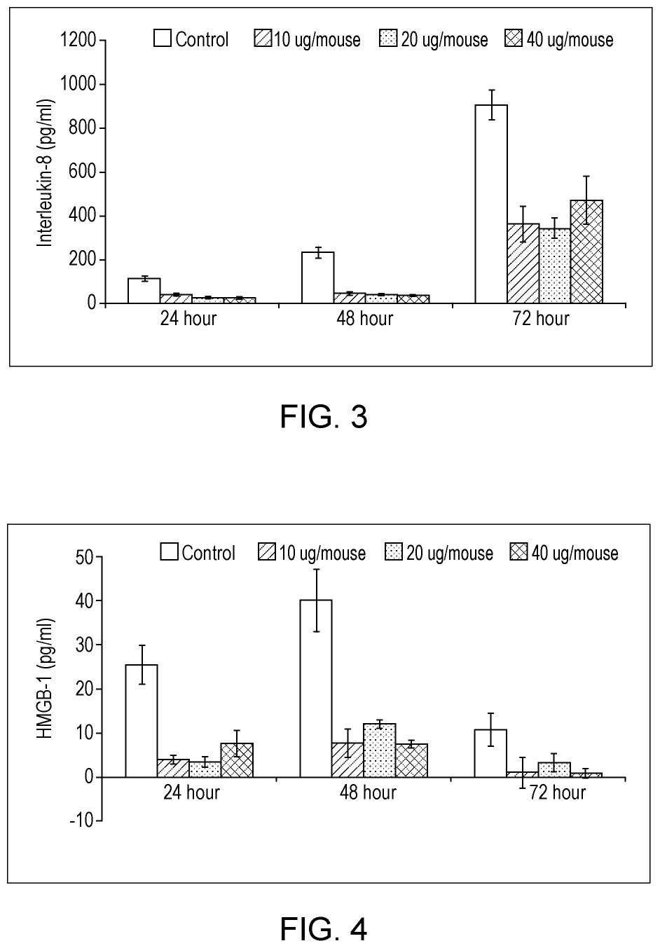

Male 7-week-old C57BL/6 mice were housed in the laboratory animal center under 22 +2° C., 55%±10% humidity, and 12 h dark/light cycle conditions. All of the mice were randomized before the experiment. The model was established by intratracheal (IT) instillation of 0.2 IU porcine pancreatic elastase (Sigma-Aldrich, St. Louis, MO, USA) using a microsprayer aerosolizer (Penn-Century, Wyndmoor, PA, USA) under general anesthesia with 3% isoflurane using a rodent anesthesia machine. Prior to administration of elastase, 10, 20 or 40 ug of sulforaphane was administered along with Curcumin (10 mg/kg), EGCG (40 mg/kg) to the mice by gavage daily for 1 week. Animals were sacrificed at the indicated timepoints. Lungs were homogenized and assessed for interleukin-8 by ELISA. Results are shown in FIG. 3 and below.

| | | 10 ug/ | 20 ug/ | 40 ug/ |
|---|---|---|---|---|
| | Control | mouse | mouse | mouse |
| | 24 hours | | | |
| 1.0 | 114.0 | 32.0 | 21.0 | 23.0 |
| 2.0 | 118.0 | 34.0 | 38.0 | 23.0 |
| 3.0 | 129.0 | 49.0 | 26.0 | 21.0 |
| 4.0 | 106.0 | 43.0 | 20.0 | 29.0 |
| 5.0 | 108.0 | 49.0 | 32.0 | 34.0 |
| 6.0 | 121.0 | 48.0 | 33.0 | 32.0 |
| 7.0 | 118.0 | 37.0 | 28.0 | 23.0 |
| 8.0 | 132.0 | 43.0 | 28.0 | 29.0 |
| 9.0 | 97.0 | 34.0 | 43.0 | 22.0 |
| 10.0 | 102.0 | 43.0 | 19.0 | 19.0 |
| Average | 114.5 | 41.2 | 28.8 | 25.5 |
| STDEV | 11.4 | 6.5 | 7.9 | 5.1 |
| | 48 hours | | | |
| 1.0 | 235.0 | 43.0 | 43.0 | 33.0 |
| 2.0 | 214.0 | 43.0 | 44.0 | 34.0 |
| 3.0 | 225.0 | 45.0 | 47.0 | 43.0 |
| 4.0 | 218.0 | 54.0 | 54.0 | 34.0 |
| 5.0 | 243.0 | 46.0 | 32.0 | 38.0 |
| 6.0 | 221.0 | 45.0 | 35.0 | 35.0 |
| 7.0 | 297.0 | 47.0 | 35.0 | 39.0 |
| 8.0 | 243.0 | 46.0 | 32.0 | 34.0 |
| 9.0 | 215.0 | 48.0 | 35.0 | 36.0 |
| 10.0 | 217.0 | 45.0 | 37.0 | 34.0 |
| Average | 232.8 | 46.2 | 39.4 | 36.0 |
| STDEV | 25.1 | 3.2 | 7.3 | 3.1 |
| | 72 hours | | | |
| 1.0 | 876.0 | 355.0 | 265.0 | 436.0 |
| 2.0 | 896.0 | 352.0 | 225.0 | 434.0 |
| 3.0 | 886.0 | 324.0 | 347.0 | 437.0 |
| 4.0 | 767.0 | 375.0 | 342.0 | 437.0 |
| 5.0 | 986.0 | 397.0 | 347.0 | 544.0 |
| 6.0 | 896.0 | 346.0 | 346.0 | 754.0 |
| 7.0 | 875.0 | 326.0 | 347.0 | 346.0 |
| 8.0 | 995.0 | 332.0 | 322.0 | 432.0 |
| 9.0 | 897.0 | 475.0 | 357.0 | 467.0 |
| 10.0 | 975.0 | 347.0 | 543.0 | 436.0 |
| Average | 904.9 | 362.9 | 344.1 | 472.3 |
| STDEV | 67.4 | 45.2 | 82.1 | 109.9 |

### Example 4: Reduction of Spike Protein Induced Systemic Inflammation by Administration of Nutraceutical with Increasing Sulforaphane Concentration (HMGB-1)

Male 7-week-old C57BL/6 mice were housed in the laboratory animal center under 22 +2° C., 55%±10% humidity, and 12 h dark/light cycle conditions. All of the mice were randomized before the experiment. Animals were administered 10, 20 or 40 ug of sulforaphane along with Curcumin (10 mg/kg), EGCG (40 mg/kg) to the mice by gavage daily for one week. 10 ug of spike protein per mouse was administered. Animals were sacrificed at the indicated timepoints. Plasma was assessed for HMGB1 by ELISA. Results are shown in FIG. 4 and below.

Raw Data

| | | 10 ug/ | 20 ug/ | 40 ug/ |
|---|---|---|---|---|
| | Control | mouse | mouse | mouse |
| | 24 hours | | | |
| 1.0 | 22.0 | 2.0 | 3.0 | 4.0 |
| 2.0 | 23.0 | 3.0 | 2.0 | 3.0 |
| 3.0 | 25.0 | 4.0 | 3.0 | 3.0 |
| 4.0 | 23.0 | 3.0 | 5.0 | 4.0 |
| 5.0 | 23.0 | 3.0 | 3.0 | 4.0 |
| 6.0 | 23.0 | 4.0 | 2.0 | 3.0 |
| 7.0 | 35.0 | 5.0 | 3.0 | 4.0 |
| 8.0 | 32.0 | 6.0 | 4.0 | 5.0 |
| 9.0 | 23.0 | 4.0 | 5.0 | 3.0 |
| 10.0 | 25.0 | 5.0 | 4.0 | 43.0 |
| Average | 25.4 | 3.9 | 3.4 | 7.6 |
| STDEV | 4.4 | 1.2 | 1.1 | 12.5 |
| | 48 hours | | | |
| 1.0 | 43.0 | 9.0 | 11.0 | 7.0 |
| 2.0 | 34.0 | 8.0 | 12.0 | 6.0 |
| 3.0 | 35.0 | 9.0 | 11.0 | 7.0 |
| 4.0 | 43.0 | 7.0 | 19.0 | 8.0 |
| 5.0 | 46.0 | 8.0 | 12.0 | 8.0 |
| 6.0 | 24.0 | 7.0 | 15.0 | 7.0 |
| 7.0 | 34.0 | 6.0 | 9.0 | 8.0 |
| 8.0 | 43.0 | 7.0 | 7.0 | 9.0 |
| 9.0 | 64.0 | 8.0 | 12.0 | 7.0 |
| 10.0 | 34.0 | 7.0 | 12.0 | 8.0 |
| Average | 40.0 | 7.6 | 12.0 | 7.5 |
| STDEV | 10.7 | 1.0 | 3.2 | 0.8 |
| | 72 hours | | | |
| 1.0 | 43.0 | 11.0 | 8.0 | 11.0 |
| 2.0 | 45.0 | 12.0 | 8.0 | 12.0 |
| 3.0 | 47.0 | 13.0 | 9.0 | 13.0 |
| 4.0 | 46.0 | 11.0 | 8.0 | 12.0 |
| 5.0 | 48.0 | 13.0 | 7.0 | 11.0 |
| 6.0 | 49.0 | 8.0 | 9.0 | 11.0 |
| 7.0 | 45.0 | 9.0 | 8.0 | 12.0 |
| 8.0 | 43.0 | 7.0 | 9.0 | 11.0 |
| 9.0 | 56.0 | 9.0 | 12.0 | 14.0 |
| 10.0 | 45.0 | 9.0 | 19.0 | 12.0 |
| Average | 46.7 | 10.2 | 9.7 | 11.9 |
| STDEV | 3.8 | 2.1 | 3.5 | 1.0 |

1. A method of treating, or reducing COVID-19 infection and/or pathology associated with said infection, said method comprising: a) identifying a patient with COVID-19; and b) administering a composition containing curcumin, sulforaphane and epigallocatechin gallate (EGCG).

2. The method of claim 1, wherein said COVID-19 associated pathology is decrease in regenerative activity of type II pulmonary epithelial cells.

3. The method of claim 2, wherein said decrease said regenerative activity of said type 1 pulmonary epithelial cells is reduced proliferative activity in response to inflammatory stimuli.

4. The method of claim 3, wherein said inflammatory stimuli is mediators released after cellular necrosis.

5. The method of claim 3, wherein said inflammatory stimuli is mediators released after cellular pyroptosis.

6. The method of claim 3, wherein said inflammatory stimuli is mediators released after cellular ferroptosis.

7. The method of claim 3, wherein said inflammatory stimuli is mediators released after cellular apoptosis.

8. The method of claim 3, wherein said inflammatory stimuli is HMGB1.

9. The method of claim 3, wherein said inflammatory stimuli is TNF-alpha.

10. The method of claim 3, wherein said inflammatory stimuli is IL-1 beta.

11. The method of claim 3, wherein said inflammatory stimuli is tissue factor.

12. The method of claim 2, wherein said decrease in regenerative activity of type 2 pulmonary epithelial cells is characterized by increased fibrosis.

13. The method of claim 2, wherein said decrease in regenerative activity of type 2 pulmonary epithelial cells is characterized by increased collagen deposition.

14. The method of claim 2, wherein said decrease in regenerative activity of type 1 pulmonary epithelial cells is characterized by increased tissue inhibitor of matrix metalloproteases.

15. The method of claim 2, wherein said decrease in regenerative activity of type 1 pulmonary epithelial cells is characterized by decreased plasminogen activity.

16. The method of claim 2, wherein said decrease in regenerative activity of type 1 pulmonary epithelial cells is characterized by decreased MMP1 activity.

17. The method of claim 2, wherein said decrease in regenerative activity of type 1 pulmonary epithelial cells is characterized by decreased MMP3 activity.

18. The method of claim 2, wherein said decrease in regenerative activity of type 1 pulmonary epithelial cells is characterized by decreased MMP5 activity.

19. The method of claim 2, wherein said decrease in regenerative activity of type 1 pulmonary epithelial cells is characterized by decreased MMP9 activity.

20. The method of claim 2, wherein said decrease in regenerative activity of type 1 pulmonary epithelial cells is characterized by decreased MMP12 activity.
